(19) United States
(12) Patent Application Publication
(10) Pub. No.: US 2024/0408061 A1
(43) Pub. Date: Dec. 12, 2024

(54) ORAL FORMULATION

(71) Applicant: ACTIMED THERAPEUTICS LTD, Ascot, Berkshire (GB)

(72) Inventors: Frank MISSELWITZ, London (GB); James Maxwell RENNIE, London (GB); Elaine MORTON, London (GB); Robert William John HAWKES, London (GB); Robin Chandra BHATTACHERJEE, London (GB)

(21) Appl. No.: 18/700,357

(22) PCT Filed: Oct. 11, 2022
(86) PCT No.: PCT/GB2022/052567

§ 371 Date: Apr. 11, 2024

(30) Foreign Application Priority Data
Oct. 12, 2021 (GB) 2114564.4

(51) Int. Cl. A61K 31/404; A61K 9/00; A61K 9/20

(52) U.S. Cl. CPC A61K 31/404; A61K 9/0053; A61K 9/2009; A61K 9/2013; A61K 9/2054; A61K 9/2059

(57) ABSTRACT

The invention relates to a solid pharmaceutical formulation suitable for oral administration comprising: (a) an active agent which is S-pindolol or a pharmaceutically acceptable salt thereof; (b) a starch excipient in an amount of at least 15.0% by weight relative to the total weight of the formulation; and (c) a cellulose excipient in an amount of at least 30.0% by weight relative to the total weight of the formulation. Also provided is the solid pharmaceutical formulation for use in therapy, for instance in the treatment of cachexia.

### FIELD OF THE INVENTION

The invention relates to a solid oral formulation comprising S-pindolol or a salt thereof, and in particular a tablet comprising S-pindolol or a salt thereof. The invention also relates to use of the solid oral formulation in therapy, for instance to treat or prevent cachexia.

### BACKGROUND OF THE INVENTION

S-pindolol is an anabolic/catabolic transforming agent which shows β₁ receptor antagonism, partial β₂ receptor agonism and central 5-HT_{1A} antagonism. S-pindolol is also known as (−)-pindolol or S(−)-pindolol and has the systematic name (2S)-1-(1H-indol-4-yloxy)-3-(1-methylethylamino) propan-2-ol. The structure of S-pindolol is shown below.

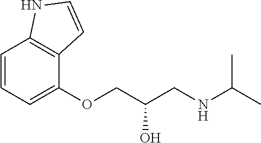 [SMILES: CC(C)NC[C@H](O)COc1cccc2[nH]ccc12]

The racemic form of S-pindolol (i.e. pindolol racemate) is described in U.S. Pat. No. 3,471,515 and is marketed for the treatment of hypertension. The treatment of wasting disorders such as cachexia and sarcopenia using enantiomerically enriched S-pindolol is described in WO 2008/068477 A1, WO 2010/125348 A1 and WO 2014/016585 A1.

S-pindolol and its salts are typically administered orally, for instance as a tablet. A tablet formulation comprising S-pindolol is described in WO 2014/016585 A1.

Formulation of pharmaceutical compounds as oral dosage forms is a complex process. Different excipients can interact differently with active agents and it is not possible to predict what these interactions and the resulting properties of the dosage form will be.

There is a need to develop an oral formulation of S-pindolol or a salt thereof which is stable during storage. It is also desirable to prepare a solid oral formulation which has a favourable dissolution profile.

### SUMMARY OF THE INVENTION

It is a surprising finding of the invention that a specific solid oral formulation comprising S-pindolol or a pharmaceutically acceptable salt thereof and significant proportions of both starch and microcrystalline cellulose as excipients is stable and has an advantageous dissolution profile.

The invention accordingly provides a solid pharmaceutical formulation suitable for oral administration comprising: (a) an active agent which is S-pindolol or a pharmaceutically acceptable salt thereof; (b) a starch excipient in an amount of at least 15.0% by weight relative to the total weight of the formulation; and (c) a cellulose excipient in an amount of at least 30.0% by weight relative to the total weight of the formulation.

The invention accordingly provides a solid pharmaceutical formulation suitable for oral administration comprising: (a) an active agent which is S-pindolol or a pharmaceutically acceptable salt thereof; (b) a starch excipient in an amount of at least 15.0% by weight relative to the total weight of the formulation; and (c) a cellulose excipient in an amount of at least 40.0% by weight relative to the total weight of the formulation.

The solid pharmaceutical formulation of the invention is typically a tablet.

Also provided by the invention is the solid pharmaceutical formulation for use in the treatment of the human or animal body.

### DETAILED DESCRIPTION OF THE INVENTION

The solid pharmaceutical formulation comprises an active agent which is S-pindolol or a pharmaceutically acceptable salt thereof. S-pindolol is preferably formulated as a salt because the free base can on occasion and under certain conditions degrade or discolour during storage.

Typically, the active agent is a pharmaceutically acceptable salt of S-pindolol. The pharmaceutically acceptable salt is typically an acid addition salt, for instance formed with an acid having a pKₐ₁ of greater than or equal to 1.5, or greater than or equal to 2.5. pKₐ₁ is the acid dissociation constant of the first proton to dissociate from the acid. For an acid having a single acidic proton, pKₐ₁ corresponds simply to the acid dissociation constant pKₐ. As used herein the pKₐ₁ values are as measured at 25° C. pKₐ and pKₐ₁ values for acids are readily available to the skilled person.

The active agent is typically a pharmaceutically acceptable salt of (i) S-pindolol and (ii) an organic acid, which organic acid has: a pKₐ₁ of greater than or equal to 1.5; and a chemical formula of CₓH_{y}(CO₂H)_{z}(OH)_{q}, where x is from 1 to 10, y is from 2 to 20, z is 1, 2 or 3 and q is 0, 1 or 2. The organic acid may, for instance, be benzoic acid, succinic acid, fumaric acid, malonic acid, acetic acid, propionic acid, glutaric acid, adipic acid, phenylacetic acid, toluic acid (including o-, m- and p-toluic acid) and naphthoic acid (including 1- and 2-naphthoic acid), citric acid or tartaric acid.

The active agent is preferably a pharmaceutically acceptable salt of (i) S-pindolol and (ii) an organic acid, which organic acid has: a pKₐ₁ of greater than or equal to 2.5; and a chemical formula of CₓH_{y}(CO₂H)_{z}, where x is from 1 to 10, y is from 2 to 20 and z is 1 or 2. The organic acid accordingly may comprise a hydrocarbyl moiety (CₓH_{y}, consisting of hydrogen and carbon) and one or two carboxylic acid groups (CO₂H). Typically x is from 2 to 7 and H is from 2 to 6. The CₓH_{y} group may be an arenyl group, an alkyl group or an alkenyl group. For instance, the CₓH_{y} group may be a divalent C₂₋₇ alkyl group, a divalent C₂₋₇ alkenyl group or a divalent phenyl group optionally substituted with one or two methyl groups.

The organic acid may, for instance, be benzoic acid, succinic acid, fumaric acid, malonic acid, acetic acid, propionic acid, glutaric acid, adipic acid, phenylacetic acid, toluic acid (including o-, m- and p-toluic acid) or naphthoic acid (including 1- and 2-naphthoic acid). Preferably, the organic acid is benzoic acid, succinic acid or fumaric acid. More preferably, the organic acid is benzoic acid or succinic acid. Typically, the active agent is S-pindolol benzoate.

The active agent may be present in an amount of from 1.0 to 45.0% by weight relative to the total weight of the formulation. The active agent is typically present in an amount of from 1.0 to 25.0% by weight relative to the total weight of the formulation, for instance from 3.0 to 25.0% by weight. Preferably, the active agent is present in an amount of from 10.0 to 20.0% by weight relative to the total weight of the formulation. For instance, the solid pharmaceutical formulation may comprise S-pindolol benzoate in an amount of from 10.0 to 20.0% by weight relative to the total weight of the formulation. The active agent may for instance be present in an amount of from 13.0 to 17.0% by weight. The active agent may alternatively be present in an amount of from 6.0 to 9.0% by weight or from 20.0 to 24.0% by weight.

The active agent may be present in an amount equivalent to 0.1 to 50 mg of S-pindolol free base. Typically, the active agent is present in an amount equivalent to from 0.5 to 20 mg of S-pindolol free base. Preferably the active agent is present an amount equivalent to from 3.0 to 15.0 mg of S-pindolol free base, more preferably in an amount equivalent to from 4.0 to 6.0 mg of S-pindolol free base. The active agent may be present in an amount equivalent to 1.0 mg, 2.5 mg, 5.0 mg, 7.5 mg, 10.0 mg, 12.5 mg or 15.0 mg of S-pindolol free base. For instance, the solid pharmaceutical formulation may comprise about 7.44 mg S-pindolol benzoate (equivalent to 5.0 mg S-pindolol free base).

The solid pharmaceutical formulation typically comprises an enantiomeric excess of S-pindolol or a pharmaceutically acceptable salt thereof (for instance an enantiomeric excess of at least 50%, at least 90% or at least 99%). Typically, the solid pharmaceutical formulation is substantially free of R-pindolol or a pharmaceutically acceptable salt thereof. For instance, the solid pharmaceutical formulation may comprise less than 1.0% by weight, less than 0.1% by weight, or less than 0.01% by weight, of R-pindolol or a pharmaceutically acceptable salt thereof relative to the total weight of the composition.

The solid pharmaceutical formulation comprises a starch excipient. The solid pharmaceutical formulation may comprise one or more starch excipients. A starch excipient is an excipient derived from starch, and may be starch or a modified starch. Starch comprises two components: amylose (typically 20 to 25% by weight) and amylopectin (typically 75 to 80% by weight). Modified starch is starch in which the hydroxyl groups have been chemically treated to alter the properties of the starch, for instance by esterification or etherification.

Preferably, the starch excipient comprises starch (which may be optionally pregelatinized as discussed below). The starch excipient typically comprises a starch obtained from a natural source. For instance, the starch excipient may comprise one or more of maize starch, wheat starch, rice starch, cassava starch and cocoyam starch. Preferably, the starch excipient comprises maize starch. Maize starch is also known as corn starch.

The starch excipient (i.e. the starch or modified starch) may additionally be pregelatinized. For instance, the starch excipient may comprise starch (such as maize starch) which is at least partially pregelatinized.

Starch which is at least partially pregelatinized corresponds to starch granules which have been suspended in water and gradually heated, causing the starch granules to absorb water, and then at least partially dried. The starch is typically partially pregelatinized. Partially pregelatinized starch is commercially available, for instance as partially pregelatinized maize starch.

The partially pregelatinized starch present in the solid pharmaceutical formulation may have a loss on drying of from 1.0 to 15.0% by weight, for instance from 5.0 to 10.0% by weight. The weight percentage of any partially pregelatinized starch present in the solid pharmaceutical formulation is based on the weight of partially pregelatinized starch present in the formulation without any additional drying (i.e. the weight of partially pregelatinized starch present added during production of the formulation).

The starch excipient is typically present in an amount of at least 20.0% by weight relative to the total weight of the formulation, for instance from 20.0 to 40.0% by weight. Preferably, the starch excipient is present in an amount of from 25.0 to 35.0% by weight relative to the total weight of the formulation. For instance, the solid pharmaceutical formulation may comprise starch or partially pregelatinized starch in an amount of from 25.0 to 30.0% by weight relative to the total weight of the formulation.

The solid pharmaceutical formulation comprises a cellulose excipient. The solid pharmaceutical formulation may comprise one or more cellulose excipients. A cellulose excipient is an excipient derived from cellulose, and may be cellulose or a modified cellulose. Cellulose is a polysaccharide formed of β(1→4) linked D-glucose units. Modified cellulose is cellulose in which the hydroxyl groups have been chemically treated to alter the properties of the cellulose, for instance by esterification or etherification. Preferably, the cellulose excipient comprises cellulose.

More preferably, the cellulose excipient comprises microcrystalline cellulose. Microcrystalline cellulose is commercially available and is partially depolymerized cellulose synthesized from α-cellulose.

The microcrystalline cellulose typically has a degree of polymerisation of less than 400 or less than 300. The microcrystalline cellulose may have a degree of polymerisation of from 200 to 250. The degree of polymerisation may be as measured by the intrinsic viscosity method (Identification B) in the United States Pharmacopoeia Monograph for microcrystalline cellulose. The method comprises determining the intrinsic viscosity, [η]_{c}, by first forming a solution by transferring 1.3 g of microcrystalline cellulose, accurately weighed to 0.1 mg, to a 125 ml conical flask; adding 25.0 mL of water and 25.0 mL of 1.0 M cupriethylenediamine hydroxide solution; immediately purging the solution with nitrogen; inserting a stopper; and shaking on a wrist action shaker until completely dissolved. An appropriate volume of the solution is transferred to a calibrated number 150 Cannon-Fenske viscosimeter. The solution is allowed to equilibrate at 25±0.1° C. for not less than 5 minutes and the intrinsic viscosity [η]_{c} is measured. The degree of polymerization, P, may be determined by the formula (95) [η]_{c}/Ws[(100−% LOD)/100], in which Ws is the weight in grams of the microcrystalline cellulose taken and % LOD is the value obtained from the test for loss on drying.

The microcrystalline cellulose typically has a particle size distribution with a D50 (median particle size) of from 80 to 160 μm, preferably from 90 to 140 μm, more preferably from 100 to 130 μm. As used herein, the D50 value is typically a Dv50 value (median particle size by volume). D50 or Dv50 values as stated herein are typically as measured by laser diffraction, for instance as measured by laser diffraction using a dry dispersion cell.

The cellulose excipient is typically present in an amount of at least 35.0% by weight relative to the total weight of the formulation or at least 40.0% by weight relative to the total weight of the formulation. The cellulose excipient is typically present in an amount of at least 45.0% by weight relative to the total weight of the formulation, for instance from 45.0 to 70.0% by weight. Preferably, the cellulose excipient is present in an amount of from 50.0 to 60.0% by weight relative to the total weight of the formulation. For instance, the solid pharmaceutical formulation may comprise from 50.0 to 60.0% by weight of microcrystalline cellulose relative to the total weight of the formulation

The solid pharmaceutical formulation typically comprises: a starch excipient which is starch (and which is preferably partially pregelatinized starch); and a cellulose excipient which is microcrystalline cellulose. The solid pharmaceutical formulation may comprise one or more further starch excipients and cellulose excipients in addition to the starch and the microcrystalline cellulose. Alternatively, the solid pharmaceutical formulation may comprise a single starch excipient which is starch and a single cellulose excipient which is microcrystalline cellulose. The solid pharmaceutical formulation may comprise a single starch excipient which is partially pregelatinized maize starch and a single cellulose excipient which is microcrystalline cellulose.

The solid pharmaceutical formulation may comprise one or more additional excipients, for instance selected from: silica; lubricants, e.g. talc, stearic acid, magnesium or calcium stearate, and/or polyethylene glycols; binding agents, e.g. arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; and wetting agents, such as lecithin, polysorbates, laurylsulphates.

The solid pharmaceutical formulation typically further comprises colloidal silica. The colloidal silica may be present in an amount of from 0.1 to 1.0% by weight relative to the total weight of the formulation. For instance, the solid pharmaceutical formulation may comprise from 0.15 to 0.35% by weight colloidal silica.

The solid pharmaceutical formulation typically further comprises a lubricant. The lubricant may for instance be one or more of talc, stearic acid, magnesium stearate or calcium stearate. The lubricant is preferably magnesium stearate. The lubricant is typically present in an amount of from 0.1 to 1.0% by weight relative to the total weight of the formulation. For instance, the solid pharmaceutical formulation may comprise from 0.15 to 0.35% by weight of a lubricant such as magnesium stearate.

Typically, the solid pharmaceutical formulation does not comprise a significant amount of lactose. For instance, the solid pharmaceutical formulation may comprise less than 10.0% by weight of lactose relative to the total weight of the formulation. The solid pharmaceutical formulation may comprise less than 10.0% by weight of lactose monohydrate. Preferably, the solid pharmaceutical formulation comprises less than 1.0% by weight of lactose. The solid pharmaceutical formulation is typically substantially free of a lactose. The solid pharmaceutical formulation typically does not comprise lactose.

Typically, the solid pharmaceutical formulation comprises: (a) 3.0 to 35.0% by weight of a pharmaceutically acceptable salt of S-pindolol as defined herein; (b) at least 20.0% by weight of a starch excipient as defined herein; and (c) at least 45.0% by weight of a cellulose excipient as defined herein, wherein the % by weight is relative to the total weight of the formulation

Typically, the solid pharmaceutical formulation comprises: (a) 5.0 to 25.0% by weight of a pharmaceutically acceptable salt of S-pindolol as defined herein; (b) at least 20.0% by weight of a starch excipient as defined herein; and (c) at least 45.0% by weight of a cellulose excipient as defined herein, wherein the % by weight is relative to the total weight of the formulation. The solid pharmaceutical formulation may comprise at least 90% by weight, at least 95% by weight, or at least 99% by weight of components (a) to (c) relative to the total weight of the composition.

The solid pharmaceutical formulation may comprise:
(a) 3 to 25% by weight of S-pindolol benzoate;
(b) 20 to 35% by weight of starch, which starch is optionally partially pregelatinized;
(c) 50 to 65% by weight of microcrystalline cellulose;
(d) optionally colloidal silica; and
(e) optionally magnesium stearate,
wherein the % by weight is relative to the total weight of the formulation.

The solid pharmaceutical formulation may comprise:
(a) 3 to 20% by weight of S-pindolol benzoate;
(b) 20 to 35% by weight of starch, which starch is optionally partially pregelatinized;
(c) 50 to 60% by weight of microcrystalline cellulose;
(d) optionally colloidal silica; and
(e) optionally magnesium stearate,
wherein the % by weight is relative to the total weight of the formulation.

The solid pharmaceutical formulation may for instance comprise:
(a) 10 to 20% by weight of S-pindolol benzoate;
(b) 20 to 30% by weight of starch, which starch is optionally partially pregelatinized;
(c) 50 to 60% by weight of microcrystalline cellulose;
(d) optionally colloidal silica; and
(e) optionally magnesium stearate,
wherein the % by weight is relative to the total weight of the formulation.

The solid pharmaceutical formulation may comprise at least 90% by weight of components (a) to (e) relative to the total weight of the composition. For instance, the solid pharmaceutical formulation may comprise at least 95% by weight, or at least 99% by weight, of components (a) to (e) relative to the total weight of the composition. The solid pharmaceutical formulation may consist, or consist essentially of, components (a) to (e).

The solid pharmaceutical formulation may be manufactured by standard formulation methods, for example by mixing, granulating or tableting the components present in the composition.

The solid pharmaceutical formulation is typically in the form of a tablet, a capsule or granules. The solid pharmaceutical formulation is preferably in the form of a tablet.

The solid pharmaceutical formulation may for instance be a tablet comprising:
(a) 6 to 24% by weight of S-pindolol benzoate;
(b) 22 to 32% by weight of maize starch,
(c) 50 to 63% by weight of microcrystalline cellulose;
(d) 0.1 to 0.5% by weight of colloidal silica; and
(e) 0.1 to 0.5% by weight of magnesium stearate,
wherein the % by weight is relative to the total weight of the formulation.

The solid pharmaceutical formulation may for instance be a tablet comprising:
(a) 10 to 20% by weight of S-pindolol benzoate;
(b) 22 to 32% by weight of maize starch,
(c) 50 to 60% by weight of microcrystalline cellulose;
(d) 0.1 to 0.5% by weight of colloidal silica; and
(e) 0.1 to 0.5% by weight of magnesium stearate,
wherein the % by weight is relative to the total weight of the formulation.

The solid pharmaceutical formulation may for instance be a tablet comprising:
(a) 10 to 20% by weight of S-pindolol benzoate;
(b) 22 to 30% by weight of maize starch,
(c) 50 to 60% by weight of microcrystalline cellulose;
(d) 0.1 to 0.5% by weight of colloidal silica; and
(e) 0.1 to 0.5% by weight of magnesium stearate,
wherein the % by weight is relative to the total weight of the formulation.

The solid pharmaceutical formulation may for instance be a tablet comprising:
(a) 10 to 20% by weight of S-pindolol benzoate;
(b) 22 to 30% by weight of partially pregelatinized maize starch,
(c) 50 to 60% by weight of microcrystalline cellulose;
(d) 0.1 to 0.5% by weight of colloidal silica; and
(e) 0.1 to 0.5% by weight of magnesium stearate,
wherein the % by weight is relative to the total weight of the formulation.

The solid pharmaceutical formulation may for instance be a tablet comprising:
(a) 13 to 17% by weight of S-pindolol benzoate;
(b) 24 to 30% by weight of partially pregelatinized maize starch,
(c) 54 to 58% by weight of microcrystalline cellulose;
(d) 0.15 to 0.35% by weight of colloidal silica; and
(e) 0.15 to 0.35% by weight magnesium stearate,
wherein the % by weight is relative to the total weight of the formulation.

The solid pharmaceutical formulation may for instance be a tablet comprising:
(a) 6 to 9% by weight of S-pindolol benzoate;
(b) 28 to 33% by weight of partially pregelatinized maize starch,
(c) 59 to 63% by weight of microcrystalline cellulose;
(d) 0.15 to 0.35% by weight of colloidal silica; and
(e) 0.15 to 0.35% by weight magnesium stearate,
wherein the % by weight is relative to the total weight of the formulation.

The solid pharmaceutical formulation may for instance be a tablet comprising:
(a) 20 to 24% by weight of S-pindolol benzoate;
(b) 23 to 28% by weight of partially pregelatinized maize starch,
(c) 49 to 54% by weight of microcrystalline cellulose;
(d) 0.15 to 0.35% by weight of colloidal silica; and
(e) 0.15 to 0.35% by weight magnesium stearate,
wherein the % by weight is relative to the total weight of the formulation.

The tablet may be produced by any standard tabletting technique. Preferably, the tablet is produced by direct compression of a powder blend of the components. The tablet may be obtainable by direct compression of a powder blend of the components, i.e. a powder blend of components (a) to (c) and optionally (d) and (e).

### Medical Uses

The solid pharmaceutical formulation is useful in the treatment or prevention of a disease or condition selected from cachexia, sarcopenia, a neuromuscular disorder, muscle weakness, hypertension, heart failure, atrial fibrillation, heart attack, angina pectoris, glaucoma and anxiety. Typically the disease or condition is selected from cachexia and muscle weakness.

The cachexia may be caused by an underlying condition. For instance, the cachexia may be caused by cancer, heart failure, chronic obstructive pulmonary diseases (COPD), liver failure, kidney failure, stroke, rheumatoid arthritis, severe burn injury or HIV/AIDS. The muscle weakness may be caused by an underlying condition. For instance, the muscle weakness may be caused by trauma, musculoskeletal injury, surgery or immobilization. The muscle weakness may be intensive care unit acquired weakness (ICUAW). The neuromuscular disorder may for instance be amyotrophic lateral sclerosis.

The invention also provides a method of treating or preventing a disease or condition selected from cachexia, sarcopenia, a neuromuscular disorder, muscle weakness, hypertension, heart failure, atrial fibrillation, heart attack, angina pectoris, glaucoma and anxiety in a subject, the method comprising administering a therapeutically effective amount of the solid pharmaceutical formulation to the subject.

Also provided by the invention is use of the solid pharmaceutical formulation in the manufacture of a medicament for the treatment or prevention of a disease or condition selected from cachexia, sarcopenia, a neuromuscular disorder, muscle weakness, hypertension, heart failure, atrial fibrillation, heart attack, angina pectoris, glaucoma and anxiety.

The pharmaceutically solid pharmaceutical formulation is typically administered orally.

An effective amount of the solid pharmaceutical formulation typically comprises an amount of S-pindolol or a pharmaceutically acceptable salt thereof equivalent to from 0.1 to 1000 mg of S-pindolol free base for a single dose. For instance, a single dose of the solid pharmaceutical formulation may comprise an amount of S-pindolol or a pharmaceutically acceptable salt thereof equivalent to from 2.5 to 50 mg or from 80 to 160 mg of S-pindolol free base. A single dose may be an amount of S-pindolol or a pharmaceutically acceptable salt thereof equivalent to from 2.5 to 15 mg of S-pindolol free base. The dose may be administered once, twice or three times a day. The dose may comprise one, two or three of the solid pharmaceutical formulations (e.g. one, two of three tablets).

The following Examples illustrate the invention.

### EXAMPLES

### Example 1—Tablet Formulations

Three tablet formulations (Formulations A, B and C) comprising S-pindolol benzoate were produced having the formulations shown in Tables 1, 2 and 3 below.

The tablets were made by direct compression of a powder blend of the components.

TABLE 1
Formulation A

| | Dose | 10,000 tablet quantity | Relative |
|---|---|---|---|
| Ingredient | (mg/tablet) | weight (g) | amounts (%) |
| S-pindolol benzoate | 7.439 | 74.39 | 14.88 |
| Starch 1500 | 14.103 | 141.03 | 28.21 |
| MCC 102 | 28.208 | 282.08 | 56.42 |
| Colloidal silica | 0.125 | 1.25 | 0.25 |
| Magnesium stearate | 0.125 | 1.25 | 0.25 |
| Total | 50.000 | 500.00 | 100.00 |

Starch 1500 is partially pregelatinized maize starch.
MCC 102 is microcrystalline cellulose.

TABLE 2
Formulation B

| | Dose | 10,000 tablet quantity | Relative |
|---|---|---|---|
| Ingredient | (mg/tablet) | weighted (g) | amounts (%) |
| S-pindolol benzoate | 7.439 | 74.39 | 14.88 |
| StarTab | 42.311 | 423.11 | 84.62 |
| Colloidal silica | 0.125 | 1.25 | 0.25 |
| Magnesium stearate | 0.125 | 1.25 | 0.25 |
| Total | 50.000 | 500.00 | 100.00 |

StarTab is a directly compressible starch mixture comprising 99% pregelatinized corn starch and 1% colloidal silicon dioxide.

TABLE 3
Formulation C

| | Dose | 10,000 tablet quantity | Relative |
|---|---|---|---|
| Ingredient | (mg/tablet) | weight (g) | amounts (%) |
| S-pindolol benzoate | 7.439 | 74.39 | 14.88 |
| Prosolv EasyTab SP | 42.561 | 425.61 | 85.12 |
| Total | 50.000 | 500.00 | 100.00 |

Prosolv EasyTab comprises 95.0-98.0% microcrystalline cellulose, 1.5-2.5% colloidal silicon dioxide, 0.5-2.0% sodium starch glycolate and 0.3-1.0% sodium stearyl fumarate. The components are agglomerated as a homogenous composite.

### Example 2—Dissolution Testing

The dissolution profiles of the three tablet formulations were assessed over 15 minutes in 0.1 M hydrochloric acid. The results are shown in Table 4.

TABLE 4

| | Dissolution (% released) | | |
|---|---|---|---|
| Vessel | Formulation A | Formulation B | Formulation C |
| 1 | 98 | 97 | 92 |
| 2 | 97 | 65 | 91 |
| 3 | 100 | 61 | 97 |
| 4 | 99 | 93 | 95 |
| 5 | 99 | 97 | 91 |
| 6 | 100 | 99 | 98 |
| Min | 97 | 61 | 91 |
| Max | 100 | 99 | 98 |
| Average | 99 | 85 | 95 |

Formulation A had a consistently higher dissolution rate than Formulation B or Formulation C.

Formulation A had a rapid disintegration time of 15 seconds. Formulations B and C had disintegration times of 27 seconds and 30 seconds respectively.

### Example 3—Stability Testing

The stability of Formulations A and C was assessed over six months during storage at conditions of 25° C./60% RH or 40° C./75% RH.

Formulation A was observed to perform well under both storage conditions for all stability characteristics studied. Formulation A also maintained a dissolution rate of at least 97% after 15 minutes for the period of the stability assessment at 25° C./60% RH.

Formulation C gave lower assay results for the active than Formulation A and this remained so during the stability assessment.

The friability of the tablets was assessed. Formulation A was found to have excellent friability (0.0%).

### CONCLUSION

Of the three tablet formulations assessed, Formulation A was found to have the most advantageous dissolution and stability characteristics.

### Example 4

Tablets of strength 5 mg, 10 mg and 15 mg S-pindolol equivalent were produced having the compositions in Table 5. The tablets were manufactured by blending and compressing the components. Starch 1500 is partially pregelatinized maize starch. MCC 102 is microcrystalline cellulose.

TABLE 5

| | 5 mg tablet | | 10 mg tablet | | 15 mg tablet | |
|---|---|---|---|---|---|---|
| | % w/w | mg/tab | % w/w | mg/tab | % w/w | mg/tab |
| S-pindolol benzoate | 7.44 | 7.44 | 14.88 | 14.88 | 22.32 | 22.32 |
| Starch 1500 | 30.69 | 30.69 | 28.21 | 28.21 | 25.73 | 25.73 |
| MCC 102 | 61.37 | 61.37 | 56.41 | 56.41 | 51.45 | 51.45 |
| Colloidal silica | 0.25 | 0.25 | 0.25 | 0.25 | 0.25 | 0.25 |
| Magnesium stearate | 0.25 | 0.25 | 0.25 | 0.25 | 0.25 | 0.25 |
| Total | 100.0 | 100.0 | 100.0 | 100.0 | 100.0 | 100.0 |

All tablets showed rapid disintegration and dissolution and had good friability characteristics.

1. A solid pharmaceutical formulation suitable for oral administration comprising:
(a) an active agent which is S-pindolol or a pharmaceutically acceptable salt thereof;
(b) a starch excipient in an amount of at least 15.0% by weight relative to the total weight of the formulation; and
(c) a cellulose excipient in an amount of at least 30.0% by weight relative to the total weight of the formulation.

2. A solid pharmaceutical formulation according to claim 1, which solid pharmaceutical composition comprises:
(a) an active agent which is S-pindolol or a pharmaceutically acceptable salt thereof;
(b) a starch excipient in an amount of at least 15.0% by weight relative to the total weight of the formulation; and
(c) a cellulose excipient in an amount of at least 40.0% by weight relative to the total weight of the formulation.

3. A solid pharmaceutical formulation according to claim 1, wherein the active agent is a pharmaceutically acceptable salt of S-pindolol.

4. A solid pharmaceutical formulation according to claim 1, wherein the active agent is a pharmaceutically acceptable salt of (i) S-pindolol and (ii) an organic acid, which organic acid has: a pKₐ₁ of greater than or equal to 2.5; and a chemical formula of CₓH_{y}(CO₂H)_{z}, where x is from 1 to 10, y is from 2 to 20 and z is 1 or 2,
preferably wherein the organic acid is benzoic acid, succinic acid, fumaric acid, malonic acid, glutaric acid, adipic acid, acetic acid, propionic acid, phenylacetic acid, toluic acid or naphthoic acid.

5. A solid pharmaceutical formulation according to claim 1, wherein the active agent is S-pindolol benzoate.

6. A solid pharmaceutical formulation according to claim 1, wherein the active agent is present in an amount of from 1.0 to 25.0% by weight relative to the total weight of the formulation,
preferably wherein the active agent is present in an amount from 10.0 to 20.0% by weight relative to the total weight of the formulation.

7. A solid pharmaceutical formulation according to claim 1, wherein the starch excipient is present in an amount of at least 20.0% by weight relative to the total weight of the formulation,
preferably wherein the starch excipient is present in an amount of from 25.0 to 35.0% by weight relative to the total weight of the formulation.

8. A solid pharmaceutical formulation according to claim 1, wherein the starch excipient comprises starch,
preferably wherein the starch excipient comprises one or more of maize starch, wheat starch, rice starch, cassava starch and cocoyam starch,
more preferably wherein the starch excipient comprises maize starch.

9. A solid pharmaceutical formulation according to claim 1, wherein the starch excipient comprises a starch which is at least partially pregelatinized.

10. A solid pharmaceutical formulation according to claim 1, wherein the cellulose excipient is present in an amount of at least 45.0% by weight relative to the total weight of the formulation,
preferably wherein the cellulose excipient is present in an amount of from 50.0 to 60.0% by weight relative to the total weight of the formulation.

11. A solid pharmaceutical formulation according to claim 1, wherein the cellulose excipient comprises microcrystalline cellulose.

12. A solid pharmaceutical formulation according to claim 1, wherein the solid pharmaceutical formulation comprises: a starch excipient which is starch; and a cellulose excipient which is microcrystalline cellulose.

13. A solid pharmaceutical formulation according to claim 1, wherein the solid pharmaceutical formulation further comprises colloidal silica,
optionally wherein the colloidal silica is present in an amount of from 0.1 to 1.0% by weight relative to the total weight of the formulation.

14. A solid pharmaceutical formulation according to claim 1, wherein the solid pharmaceutical formulation further comprises a lubricant, preferably wherein the lubricant is magnesium stearate,
optionally wherein the lubricant is present in an amount of from 0.1 to 1.0% by weight relative to the total weight of the formulation.

15. A solid pharmaceutical formulation according to claim 1, wherein the solid pharmaceutical formulation comprises less than 10.0% by weight of lactose relative to the total weight of the formulation,
preferably wherein the solid pharmaceutical formulation comprises less than 1.0% by weight of lactose.

16. A solid pharmaceutical formulation according to claim 1, wherein the solid pharmaceutical formulation comprises:
(a) 5.0 to 25.0% by weight of a pharmaceutically acceptable salt of S-pindolol;
(b) at least 20.0% by weight of a starch excipient, preferably wherein the starch excipient is present in an amount of from 25.0 to 35.0% by weight; and
(c) at least 45.0% by weight of a cellulose excipient, preferably wherein the cellulose excipient is present in an amount of from 50.0 to 60.0% by weight, wherein the % by weight is relative to the total weight of the formulation.

17. A solid pharmaceutical formulation according to claim 1,
wherein the solid pharmaceutical formulation comprises:
(a) 10 to 20% by weight of S-pindolol benzoate;
(b) 20 to 30% by weight of starch;
(c) 50 to 60% by weight of microcrystalline cellulose;
(d) optionally colloidal silica; and
(e) optionally magnesium stearate,
wherein the % by weight is relative to the total weight of the formulation.

18. A solid pharmaceutical formulation according to claim 1, wherein the solid pharmaceutical formulation is a tablet.

19. A solid pharmaceutical formulation according to claim 1, wherein the solid pharmaceutical formulation is a tablet comprising:
(a) 10 to 20% by weight of S-pindolol benzoate;
(b) 22 to 30% by weight of maize starch, which maize starch is preferably partially pregelatinized;
(c) 50 to 60% by weight of microcrystalline cellulose;
(d) 0.1 to 0.5% by weight of colloidal silica; and
(e) 0.1 to 0.5% by weight of magnesium stearate,
wherein the % by weight is relative to the total weight of the formulation.

20. (canceled)

21. (canceled)

22. (canceled)

23. A method of treating or preventing a disease or condition selected from cachexia, sarcopenia, a neuromuscular disorder, muscle weakness, hypertension, heart failure, atrial fibrillation, heart attack, angina pectoris, glaucoma and anxiety in a subject, the method comprising administering to the subject a therapeutically effective amount of a solid pharmaceutical formulation according to claim 1.
